(19) United States
(12) Patent Application Publication
(10) Pub. No.: US 2026/0024653 A1
(43) Pub. Date: Jan. 22, 2026

(54) RULE BASED DATA ITEM TRAVERSAL TECHNICAL FIELD

(71) Applicant: Community Care Health Network, LLC, Nashville, TN (US)

(72) Inventors: Sowri Krishnan, Scottsdale, AZ (US); Matthew Walley, Scottsdale, AZ (US)

(21) Appl. No.: 18/775,788

(22) Filed: Jul. 17, 2024

(51) Int. Cl. G16H 40/20; G06F 9/451; G16H 10/60

(52) U.S. Cl. CPC G16H 40/20; G06F 9/451; G16H 10/60

(57) ABSTRACT

Methods, systems, and computer-readable storage media for resource allocation actions. An inquiry to render a user interface with first visual representations of upcoming events is received, from a user device. The inquiry includes an identifier used to retrieve first data items specifying first user qualifications. Second data items specifying user attributes and labeled with an indicator representing a location, are looked-up, in the memory. The second data items are filtered to identify filtered data items by matching attributes with the qualifications relative to the location. The filtered data items are transmitted, to the user interface module, to generate the user interface comprising first visual representations of upcoming events within the location. An indication of a selected visual representation is received, through the user interface from the user device. Second data items stored in the memory are automatically traversed to update the user interface.

### TECHNICAL FIELD

The present disclosure relates to data item traversal rules. More particularly, implementations of the present disclosure are directed to rule based resource allocation actions.

### BACKGROUND

Data item traversal rules are important for reliably processing sequences of actions. For example, the data item traversal rules can be applied to validate conditions in a section of a dataset, relative to a particular data type, range, or format. The data item traversal rules can present a set of problems and limitations. Some challenges of the data item traversal rules are related to rule subjectivity, the complexity of rules, dynamic interdependencies of data analyzed by rules, cost and resources, data integration, and others. The correction of resource allocation actions identified at being risk of failure of completion, using the data item traversal rules, can also raise multiple challenges. These challenges could limit implementations of solutions aimed at providing corrected resource allocation actions-especially if the dataset included multiple problems, or if the information related to the failure to complete an action is incomplete.

### SUMMARY

Implementations of the present disclosure are directed to data item traversal rules. More particularly, implementations of the present disclosure are directed to rule based resource allocation actions automatically triggered by identification of filtered data items.

In some implementations, a computer-implemented method includes receiving, from a user device, an inquiry to render a user interface with the first visual representations of upcoming events, the inquiry specifying an identifier of a first user associated with the user device and the inquiry specifying a location of the user device, retrieving, from a memory, one or more first data items associated with the identifier of the first user associated with the user device transmitting the inquiry, with the one or more first data items specifying one or more qualifications of the first user, looking-up, in the memory, the second data items, each of the second data items being labeled with an indicator representing a location, the second data items representing upcoming events of second users, and each of the second data items specifying one or more attributes of a respective second user, filtering the second data items to identify filtered data items by matching the one or more attributes with the one or more qualifications relative to the location, transmitting, to the user interface module, the filtered data items, to generate the user interface including first visual representations of upcoming events within the location, receiving, through the user interface from the user device, an indication of a selected visual representation from the first visual representations, the indication specifying the identifier of the first user of the user device, identifying a portion of the filtered data items represented by the selected visual representation, generating, in memory, an association between the portion of the filtered data and the identifier of the first user, the association specifying that an accepted event of the first user represented by the identifier associated with the one or more first data items, and transmitting, to the user interface module, the association specifying the accepted event, to traverse the second data items stored in the memory to automatically update the user interface rendered on the user device with a second visual representation representing the accepted event, the second visual representation being rendered in real-time relative to the indication of the selected visual representation.

The foregoing and other implementations can each optionally include one or more of the following features, alone or in combination. In particular, implementations can include one or more of the following features:

In a first aspect, combinable with any of the previous aspects, the one or more first data items include medical qualifications and availability calendars relative to the location. In another aspect, combinable with any of the previous aspects, the one or more second data items include medical records and appointment zone data. In another aspect, combinable with any of the previous aspects, matching the one or more attributes with the one or more qualifications relative to the location includes mapping the medical records and the medical qualifications relative to zone mapping based on the location. In another aspect, combinable with any of the previous aspects, the mapping defines associations between the appointment zone data and the location that are compatible with the availability calendars, wherein the mapping includes executing a geo-dialing algorithm determining distances and estimated travel times between the appointment zone data and the location. In another aspect, combinable with any of the previous aspects, the respective available upcoming events are ranked based on a timeline. In another aspect, combinable with any of the previous aspects, the computer-implemented method further includes: receiving event data of an impeding event affecting the accepted event, updating the user interface to display corrected entries indicative of the impeding event, wherein the impeding event affecting the accepted event includes a traffic event associated with a predicted failure to complete the accepted event by the first user, identifying a remedy to compensate for the impeding event, generating a recommendation including a permutation of a traversal of the second data items, and generating a notification for displaying the recommendation. In another aspect, combinable with any of the previous aspects, the filtered data items are encrypted for transmission.

These and other implementations can each optionally include one or more of the following advantages. The described resource allocation actions are integrated with the rules instead of being a disconnected step, such that data collection is automatically triggered leading to an optimization of resource allocation actions. The described approach can implement powerful data item filtering and best traversal functions for resource allocation actions, which minimizes or eliminates corrupted and inconsistent resource allocation actions. Remediation data preparation replacement steps of the described implementations can be auto generated from resource allocation actions. Remediation data can trigger automated system redirection for adapting to real time factors for completion of resource distribution according to imposed limitations. Resource allocation actions, as described, are less error prone, being tailored to predict and proactively prevent undue delays, when implementing a remediation plan. Resource allocation actions leverage outputs of prediction models based on artificial intelligence to suggest previous resource allocation actions for new action sequences with similar constraints and structures. The described prediction models can also be leveraged to generate the rule remediation action definitions for optimization of action correction processes.

It is appreciated that methods in accordance with the present disclosure can include any combination of the aspects and features described herein. That is, methods in accordance with the present disclosure are not limited to the combinations of aspects and features specifically described herein, but also include any combination of the aspects and features provided.

The details of one or more implementations of the present disclosure are set forth in the accompanying drawings and the description below. Other features and advantages of the present disclosure will be apparent from the description and drawings, and from the claims.

### DESCRIPTION OF DRAWINGS

FIG. 1A depicts an example system in accordance with implementations of the present disclosure.

FIG. 1B depicts another example system in accordance with implementations of the present disclosure.

FIG. 2A is a conceptual diagram of locations of service receivers and a service provider in accordance with implementations of the present disclosure.

FIG. 2B is an example of a schedule in accordance with implementations of the present disclosure.

FIG. 2C is an example of an action sequence in accordance with implementations of the present disclosure.

FIG. 3A depicts an example of a display panel for data item selection, in accordance with implementations of the present disclosure.

FIG. 3B depicts an example display panel of action selection that can be executed in accordance with implementations of the present disclosure.

FIG. 3C depicts an example display panel of a selectable action that can be displayed in accordance with implementations of the present disclosure.

FIG. 4A depicts an example display panel of route selection for resource allocation actions that can be executed in accordance with implementations of the present disclosure.

FIG. 4B depicts another example display panel of route planning for resource allocation actions that can be executed in accordance with implementations of the present disclosure.

FIG. 4C depicts an example display panel of route mapping for resource allocation actions that can be executed in accordance with implementations of the present disclosure.

FIG. 4D depicts an example display panel of communication selection during route planning for resource allocation actions that can be executed in accordance with implementations of the present disclosure.

FIG. 5A depicts a flow chart of an example processes for resource allocation actions that can be executed in accordance with implementations of the present disclosure.

FIG. 5B depicts a flow chart of an example processes for resource allocation actions that can be executed in accordance with implementations of the present disclosure.

FIG. 6 is a schematic illustration of example computer systems that can be used to execute implementations of the present disclosure.

Like reference symbols in the various drawings indicate like elements.

### DETAILED DESCRIPTION

Implementations of the present disclosure are directed to data item and actions traversal rules. More particularly, implementations of the present disclosure are directed to resource allocation actions automatically triggered by identification of data entries, potentially affecting a resource allocation action. The traversal and/or replacement resource allocation actions can be implemented according to rule definitions. The rule definitions define simple or complex traversal actions and/or replacements of resource allocation actions to automatize traversal of a sequence of actions that is updated according to ongoing conditions. The traversal of data items can include a combination of multiple replacement resource allocation actions defined in a remediation plan. The traversal of data items stored in the memory can trigger an automatic update of a user interface rendered on a user device, the visual representation being rendered in real-time relative to an indication of an ongoing resource allocation action.

Traditionally, a user evaluates the information about the reason for failure of a resource allocation action and attempts to correct an action sequence by updating the action sequence. The correction of action sequences can be disrupted by coexistent problems, or if the information about the reason of action failure or evaluation of replacement options is incomplete. For example, the correction of erroneous entries of the action sequences can include replacement of a failed action with another action, replacement of one system allocation with another system allocation, replacement of an action with another from the same sequence or another action sequence from another system allocation. Generating a sequence of adequate corrections can be a challenging task. For example, if the remediations are not connected to the rules, it is complicated to generate a correct workflow of action replacements.

Addressing the limitations of traditional traversal and correction mechanisms of sequences of actions, the approach described in the present disclosure provides secure, optimized, and consistent traversal of data items using prediction models. The prediction models can predict remediation possibilities of a sequence of rule actions including repeatable automated corrections based on several remediation factors. The remediation factors include usage tracking, content type associations with resource allocation actions, and replacement of common data errors that the system recognizes. The prediction models can optimize the traversal of data items in a sequence of actions and the remediation workflow in terms of efficiency of data processing and accuracy of results. The optimization of the traversal of data items in a sequence of actions can further include automatic activation of systems to execute resource allocation actions.

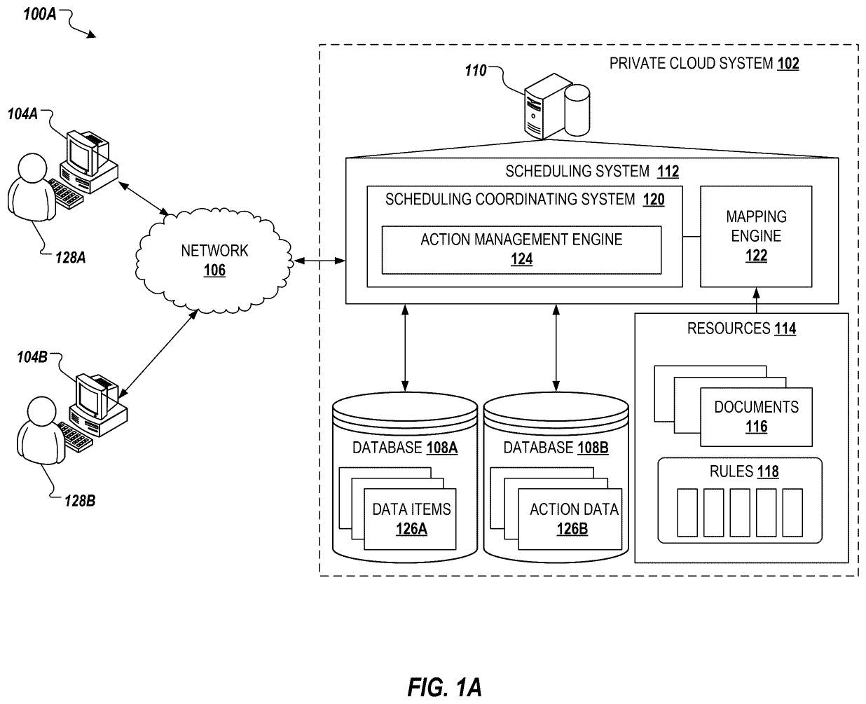

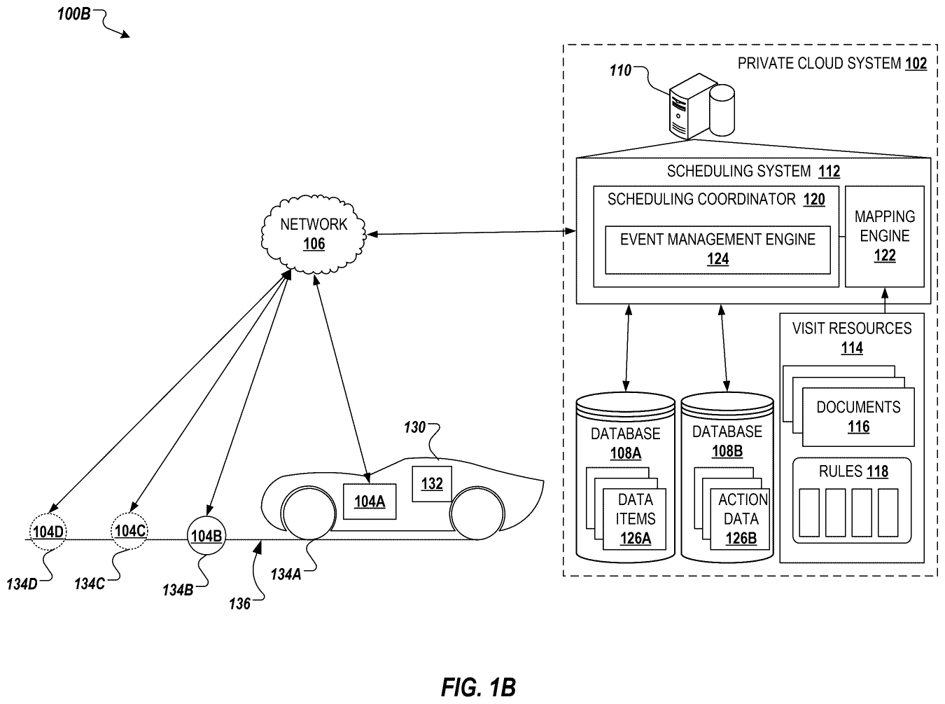

The described approach can be implemented in multiple technological fields, including, for example, scheduling of medical appointments. Within context examples, a user device of an entity (e.g., medical practitioner or service provider) can receive a request to render a user interface with first visual representations of requested actions (e.g., medical visits or service-related actions). The request can include an identifier of the entity associated with the user device and can specify a geographic location of the user device. The identifier of the entity can be used to identify data items associated with the identifier. The data items can be filtered, by a scheduling system, and encrypted for transmission to generate the requested user interface displaying the sequence of actions (e.g., medical visits or service-related actions) in the requested geographic location. An action acceptance of a data item can trigger an acquisition of data (e.g., sensor data) associated with the respective action to verify completion or potential failure to complete an action for traversing the sequence of actions. If one of the actions is at risk of potential failure, the rule actions can provide auto suggestions for potential remediations, such as transformations to remediate the actions before proceeding to a subsequent step of the sequence of actions. The remediation transformations can also be visual data driven ways to build and test data entry transformations with a small subset of data. During a training phase of the prediction models, some remediation transformations can be marked as “favorite” such remedial actions and can be saved as templates for future use in connection to mapped remediation rules. The remediation workflow can include application of weights to the rule actions for determining an optimized sequence of resource allocation actions. For example, when remediation rules are created, marked weightage can be used for the severity of a remediation failure and the corresponding level of severity and urgency can be reflected in the rule actions. Further advantages of the remediation workflow are described in detail with reference to FIGS. 1-6.

FIG. 1A depicts an example system 100A in accordance with implementations of the present disclosure. In the depicted example, the example system 100A includes a private cloud system 102, one or more user devices 104A, 104B, and a network 106. Although shown separately, in some implementations, functionality of two or more components of the example system 100 or the private cloud system 102 can be provided by a single system or server. In some implementations, the functionality of one illustrated example system 100, server, or component can be provided by multiple systems, servers, or components, respectively.

The private cloud system 102 includes one or more databases (e.g., processors, memory) 1008A, 108B, one or more server devices 110 hosting a scheduling system 112, and resources 114 including one or more documents 116 and one or more rules 118. The scheduling system 112 includes a scheduling coordinating system 120 and a mapping engine 122. The scheduling coordinating system 120 includes an action management engine 124.

In the depicted example, a user 128A interacts with an application executed (as a service) by the user device 104A that accesses data items 126A stored by the database 108A to schedule a sequence of actions, for which action data 126B are collected and stored by the database 108B. The data item 126A can include data provided by one or more users 128B as a user input received by the user device 104B, transmitted for storage to the private cloud system 102. In some examples, the user devices 104A, 104B include any appropriate type of computing device such as a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or an appropriate combination of any two or more of these devices or other data processing devices. In some examples, the user devices 104A, 104B can communicate with the private cloud system 102 over the network 106.

In some implementations, the network 106 can include a large computer network, such as a local area network (LAN), a wide area network (WAN), the Internet, a cellular network, a telephone network (e.g., PSTN) or an appropriate combination thereof connecting any number of communication devices, mobile computing devices, fixed computing devices and server systems.

In the example of FIG. 1A, the server device 110 is intended to represent various forms of servers including, but not limited to a web server, an application server, a proxy server, a network server, and/or a server pool. In general, server systems accept requests for applications as services and provides such microservices to any number of user devices (e.g., the user devices 104A, 104B over the network 106).

The databases (e.g., processors, memory) 108A, 108B can include any type of database module and persistencies that can take the form of volatile and/or non-volatile memory including, without limitation, magnetic media, optical media, random access memory (RAM), read-only memory (ROM), removable media, or any other suitable local or remote memory component. The databases (e.g., processors, memory) 108A, 108B can store various data records that can be mapped to one or more documents 116 according to rules 118 for generating action sequences. For example, the databases 108A, 108B can store objects or data, including caches, classes, frameworks, applications, backup data, application objects, jobs, web pages, web page templates, database tables, database queries, repositories storing application data and/or dynamic information, and any other appropriate information including any parameters, variables, algorithms, instructions, rules, constraints, or references thereto associated with the purposes of the private cloud system 102. The databases 108A, 108B can be homogeneous such that all microservices related to an application use an in-memory database. In some implementations, the rules 118 can be mapped to data items 126A and action data 126B stored in the databases 108A, 108B (e.g., a first portion of the microservices use a first database 108A and a second portion of the microservices use a second database 108B) of different action types. Examples of heterogeneous databases 108A, 108B include specialized databases, such as graph databases, vector databases, time-series databases or other database types related to particular microservice functions. In some implementations, other forms of persistencies can be used, for example, object datastores.

The scheduling system 112 can provide scheduling and remediation commands that can verify integrity and correctness of data entries of data items 126A and action data 126B stored in the databases 108A, 108B using the resources 114 of the private cloud system 102. The resources 114 represent resources that are provisioned within the private cloud system 102 for use by the scheduling system 112. Example resources 114 can include, without limitation, documents 116, rules 118, database systems, applications, servers, physical machines, virtual machines, containers, and the like. In some examples, the scheduling and remediation mechanism for the data items 126A can be provisioned within the private cloud system 102 and can be automatically adjusted according to documents 116 or in response to remediation demands triggered by changes to action data (.e.g., in response to an error detected by the action management engine 124 monitoring actions data 126B received from the user device 104A).

The action management engine 124 processes the data items 126A to generate a sequence of actions, processes the action data 126B for each action of the action sequence according to rules 118 mapped to data items 126A, by the mapping engine 122 and generates the documents 116 for each completed or failed action of the sequence. The action management engine 124 provides an order of execution of actions within the action sequence and remedial actions to correct failed actions. An action data 126B outside a set range can be identified as a failed action that triggers the action management engine 124 to generate and execute a remediation plan including a remediation operation (e.g., action to correct the failed action). Each of the action management engine 124 and the mapping engine 122 can include a prediction model.

The prediction model can be a trainable artificial intelligence model (e.g., machine learning model). The prediction model can include various layers of a neural network process machine learning inferences by performing large quantities of computations (e.g., matrix multiplications). Computation processes performed within a neural network layer (e.g., a convolutional layer) can include multiplying an input activation (e.g., a first operand) with a weight (e.g., a second operand) on one or more cycles and performing an accumulation of data items over multiple cycles. An output activation is generated based on multiply and accumulation operations performed on the two operands.

The mapping engine 122 can include a prediction model trained to predict resource allocation actions applicable to failed actions having abnormal action data 126B based on several mapping factors (e.g., action tracking, action type associations with resource allocation actions, and replacement of failed actions that the mapping engine 122 recognizes according to mapping patterns). In other words, the trained prediction model of the mapping engine 122 can map the abnormal action data 126B of the database 108B to remediation operations that can be provided as input features for the training of the machine learning model. For example, the mapping engine 122 can convert each abnormal action data 126B and associated data items 126A to predict remediation operations that can replace the failed actions with possible actions of the same sequence of actions or another sequence of actions associated with another user identifier. During a training phase of the prediction models of the mapping engine 122, some remediation operations can be marked as “favorite” and can be saved as templates for future use in connection to mapped remediation rules. The identified remediation operations can be provided by the mapping engine 122 to the action management engine 124 to generate a remediation workflow including a substitute sequence of actions that replaces the original sequence of actions.

The action management engine 124 can include a prediction model trained to predict an optimized sequence of actions in terms of efficiency of performing a set of actions according to a particular order that maximizes a chance of successful completion of all actions satisfying set restraints (e.g., geographical restrictions, resource restrictions, and/or time restrictions). The prediction model includes a machine learning model that can be trained using as input a training dataset with sequences of resource allocation actions and failed actions throughout multiple sequences of actions. The selection of an optimized sequence of actions and remediation workflow can reduce processing complexity and automatically provides consistency across use-cases by avoiding repeated replacement of a particular failed action that would be required by an incorrectly executed order of action corrections that can conflict with other actions or can generate new failed actions related to data items and action data relationships. For example, the action management engine 124 can be trained to predict an optimized sequence of actions and remediation workflow, resulting in correction of failed actions without triggering new actions. The remediation actions (or correction operations) can be provided to the training system as input features, and the operations-event characteristics contain data items that can be provided to the training system as target outputs.

The action management engine 124, during training phase, can select the type of machine learning model to be trained, e.g., pick a predefined or default type of machine learning model, or analyze the input features and the target outputs to identify a particular type of machine learning model. For example, types of machine learning models can include a gradient boosted trees model, a generalized linear model, a support vector machine, a decision tree model, or a neural network model, e.g., a multilayer perceptron (MLP). The machine learning models can be trained using machine learning training algorithms such as minimizing an error, computing a gradient, or performing backpropagation.

During a training phase of the prediction models, some action sequences and remediation workflows can be marked as “favorite” sequences of (remedial) actions and can be saved as templates for future use in connection to data items. The action sequences and remediation workflow can include application of weights to the rule actions for determining an optimized sequence of resource allocation actions. For example, when remediation rules are created, there can be user marked weightage for the severity of a remediation failure and the corresponding level of severity and urgency can be reflected in the remediation rule actions.

FIG. 1B depicts another example system 100B in accordance with implementations of the present disclosure. The example system 100B can include any component of the example system 100A and shows the user device 104A included in or connected to a vehicle 130. The vehicle 130 can execute actions in response to a trigger received from the user device 104A and/or sensor data received from a sensor 132.

In some implementations, the vehicle 130 can have autonomous capability of executing actions. As used herein, the term “autonomous capability” refers to a function, feature, or facility that enables a vehicle to be partially or fully operated without real-time human intervention, including without limitation fully autonomous (e.g., driverless) vehicles, highly autonomous vehicles, and conditionally autonomous vehicles.

The vehicle 130 can include hardware components and software components. The vehicle 130 can store data and send/receive data generated in real-time that supports the operation of the vehicle 130. The operation of the vehicle 130 includes transportation of goods and/or people from one location 134A to one or more target locations 134B, 134C, 134D that can be associated with locations of user devices 104B, 104C, 104D. The vehicle 130 can include cars, motorcycles, buses, trains, airplanes, drones, trucks, boats, ships, submersibles, dirigibles, or any other type of vehicle.

A sequence of actions executed by the vehicle 130 can include a trajectory 136 defining a path or route to navigate the vehicle 130 from a first spatiotemporal location 134A to second spatiotemporal location 134B. In an implementation, the first spatiotemporal location 134A is referred to as the initial or starting location and the second spatiotemporal location 134B is referred to as the destination, final location, target location, goal position, or goal location. In some examples, a trajectory 136 includes one or more segments (e.g., sections of road) and each segment includes one or more blocks (e.g., portions of a lane or intersection). In some implementations, the spatiotemporal locations correspond to real world locations, which within a context example include medical visit locations.

The sensor(s) 132 includes one or more hardware components that detect information about the environment surrounding the sensor 132. Some of the hardware components can include sensing components (e.g., image sensors, biometric sensors), transmitting and/or receiving components (e.g., laser or radio frequency wave transmitters and receivers), electronic components such as analog-to-digital converters, a data storage device (such as a RAM and/or a nonvolatile storage), software or firmware components and data processing components such as an ASIC (application-specific integrated circuit), a microprocessor and/or a microcontroller. Sensor data can include a scene description structured as a data structure (e.g., list) or data stream that includes one or more classified or labeled objects detected by one or more sensors on the vehicle 130 or provided by the user device 104A and/or the scheduling system 112.

In some implementations, the sensors 132 can measure or infer properties of state or condition of the vehicle 130, such as position, linear velocity and acceleration, angular velocity and acceleration, and heading (e.g., an orientation of the leading end of vehicle 130). Example of sensors 132 are inertial measurement units (IMU) that measure both vehicle linear accelerations and angular rates, wheel speed sensors for measuring or estimating wheel slip ratios, wheel brake pressure or braking torque sensors, engine torque or wheel torque sensors, and steering angle and angular rate sensors. In some implementations, the sensors 132 also include sensors for sensing or measuring properties of the environment of the vehicle 130. For example, monocular or stereo video cameras in the visible light, infrared or thermal (or both) spectra, LiDAR, RADAR, ultrasonic sensors, time-of-flight (TOF) depth sensors, speed sensors, temperature sensors, humidity sensors, and precipitation sensors.

In some implementations, the remotely located database 108B stores and transmits action data including digital data received from the vehicle 130 (e.g., storing data such as road and street locations). Such data is stored by the memory on the vehicle 130 and transmitted to the private cloud system 102 via a communications channel from the remotely located database 108B. The remotely located database 108B stores and transmits historical information about driving properties (e.g., speed and acceleration profiles) of vehicles that have previously traveled along trajectory 136 at a set frequency during an execution of a sequence of actions. The user device 104A located in and communicatively coupled to the vehicle 130 can algorithmically generate control actions based on both real-time sensor data and prior information, allowing the vehicle 130 to execute its autonomous driving capabilities to complete a sequence of actions.

Within the context example, the user devices 104B, 104C, 104D can transmit to the scheduling system 112, requests to receive services (e.g., medical visits) at particular locations 134B, 134C, 134D (e.g., medical facilities). The requests include first data items 126A identifying the user devices 104B, 104C, 104D and the users (e.g., patients) associated with the user devices 104B, 104C, 104D. With continued reference to the context example, the user device 104A coupled to the vehicle 130 can transmit to the scheduling system 112, a request to schedule offered services according to a set of conditions including temporal restrictions (e.g., beginning time and end time for particular days) geographical restrictions (e.g., geographic boundary defined as a spatial distance from a starting location 134A to a maximum goal location 134D, travel time, or road conditions) and ecological restrictions (e.g., energy or fuel consumption). The requests include second data items 126A identifying the user devices 104A and the users (e.g., medical practitioner) associated with the user devices 104A. The scheduling system 112 can process the data items 126A to generate a sequence of actions including travel to the particular locations 134B, 134C, 134D, executed by the vehicle 130, and delivery of services (e.g., medical visits including medical diagnosis executed using a medical device that can be included in the vehicle 130).

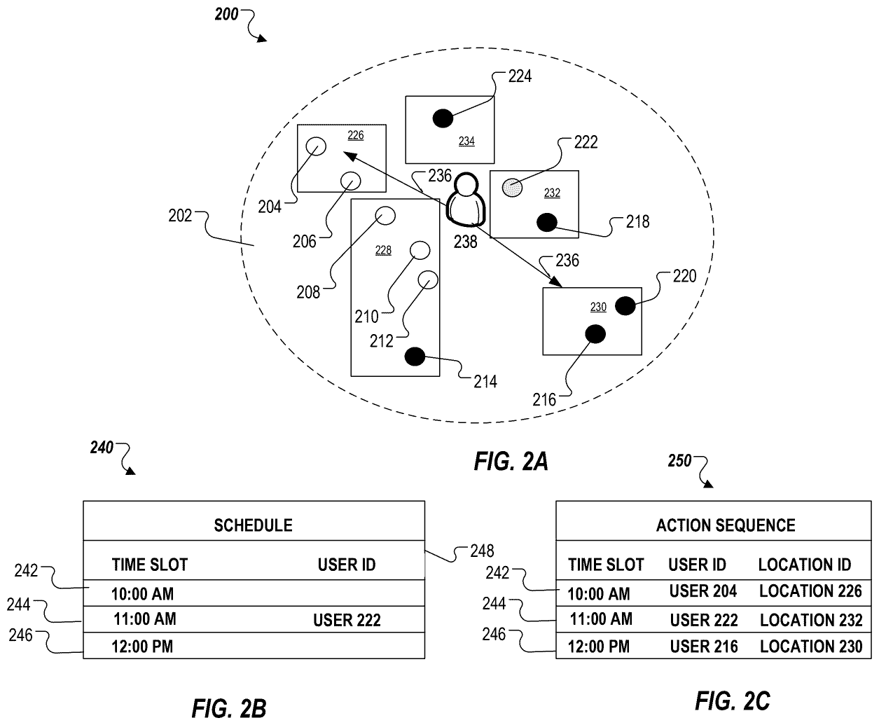

Referring to FIG. 2A, an example diagram 200 shows a boundary 202 that is representative of the geographic boundary defined around a geographic location (e.g., a starting location, such as location 134A described with reference to FIG. 1B) of a user 238 (e.g., medical practitioner) associated with a user device (e.g., the user device 104A coupled to the vehicle 130 described with reference to FIG. 1B). In the example diagram 200, the service receivers 204, 206, 208, 210, 212, 214, 216, 218, 220, and 224 include users associated with user devices (e.g., user devices 104B, 104C, 104D described with reference to FIGS. 1A and 1B) that requested services (e.g., medical appointments) at respective geographic locations, e.g., 226, 228, 230, 232, and 234 that can include zones assigned to a service (e.g., health care) user 238 by a scheduling system (e.g., scheduling system 112 described with reference to FIGS. 1A and 1B).

Generally, a user providing services includes a member who generate a user input for a user device to schedule a sequence of actions to provide services within the geographic boundary 202 by executing actions, such as traveling to a sequence of geographic locations 226, 228, 230, 232, and 234 to provide services at each of the selected geographic locations226, 228, 230, 232, and 234. In the example illustrated in FIG. 2A, the geographic locations 226, 228, 230, 232, and 234 of the service receivers 204, 206, 208, 210, 212, 214, 216, 218, 220, and 224 are positioned within the boundary 202 such that one or more of the service receivers 204, 206, 208, 210, 212, 214, 216, 218, 220, and 224 is scheduled for service at a particular location 226, 228, 230, 232, and 234. The geographic location can include a residence of the service receivers 204, 206, 208, 210, 212, 214, 216, 218, 220, and 224 or a facility configured for selected services (e.g., medical facility).

The user 238 provides (medical) services to particular users 204, 206, 216, 220 within the boundary 202 as selected by the scheduling system. Generally, a geographic zone specifies an area or a region in which the scheduling system identified that the user 238 can provide requested (medical) services that satisfy the restriction criteria imposed by the user 238.

The scheduling system can assign a radial distance 236 and an order of actions forming a sequence of actions that satisfy the restriction criteria imposed by the user 238 and reduce a travel time between the geographic locations 226, 228, 230, 232, and 234 and maximize service time within defined time limit. With the scheduling system assigned radial distances, a travel of the user 238 is restricted by the radial distance 236, even if the user requesting services is otherwise in an assigned zone of the user 238.

FIG. 2B is an example of a schedule 240 in accordance with implementations of the present disclosure. The example schedule 240 includes time slots 242, 244, 246 for a service provider. In the example illustrated in FIG. 2B, the time slot 244 is shown as being already scheduled for the user 222 and the time slots 242, 246 are empty and are available to be scheduled. To populate (e.g., schedule) time slot 242, the scheduling system retrieves a list of users that are filtered as qualifying to receive services from the user 238. In this example, the filtered users are requested services within a selected region (e.g., boundary 202) as the user 238 and have accepted the invitation to schedule the service. The scheduling system filters the users by excluding those users who are located outside of zones 226, 228, 230, 232, 234 or require services that do not meet the qualifications of the user 238. The scheduling system can further filter the users by removing those users who are located at a distance that is beyond radial distance 236, e.g., even if those users are otherwise within a zone of user 238. For the remaining (filtered) users, the scheduling system determines action plans for the selected users and determines whether user 238 present matching credentials for the respective action plans. Action plans with unmatching credentials are also removed from the list of selected users for the service user 238.

FIG. 2C is an example of an action sequence 250 in accordance with implementations of the present disclosure. The action sequence 250 can be generated by a scheduling system to include a list of actions to be performed at the time slots 242, 244, 246, at which the service provider is available to perform services. The action sequence 250 defines an order, in which users 204, 222, 216 are scheduled to receive services at respective geographical locations 226, 232, 230 within the geographic boundary 202 set by the user 238 providing services. In the illustrated example, slot 242 is the first appointment of the day for user 238. The scheduling system retrieves, from a database (e.g., database 108A described with reference to FIGS. 1A and 1B), data items (e.g., data items 126A described with reference to FIGS. 1A and 1B) including information indicative of a starting geographic location of user 238. The starting location can include a location of a residence of user 238, a location of an office of a user 238, a point of interest of the user 238 and or any other geographical location selected by the user 238. The information indicative of the starting geographic location includes a geocode of the starting location. The scheduling system retrieves, from the database, geocodes of each of the call users (e.g., the users who remain on the filtered list of call users). Using the geocodes, scheduling system executes a geo-dialing algorithm that calculates a distance (e.g., a straight-line distance) between user 238 and each of the users requesting services. The scheduling system can rank users that requested services based on their location proximity to the starting location of user 238 to prioritize the shortest distances between the user 238 and the filtered users). The scheduling system can optimize the action sequence 250 to minimize a risk of the user 238 to fail to execute the listed actions according to the scheduled chronological order 242, 244, 246, for providing services to the users 204, 222, 216 at their respective locations 226, 232, 230.

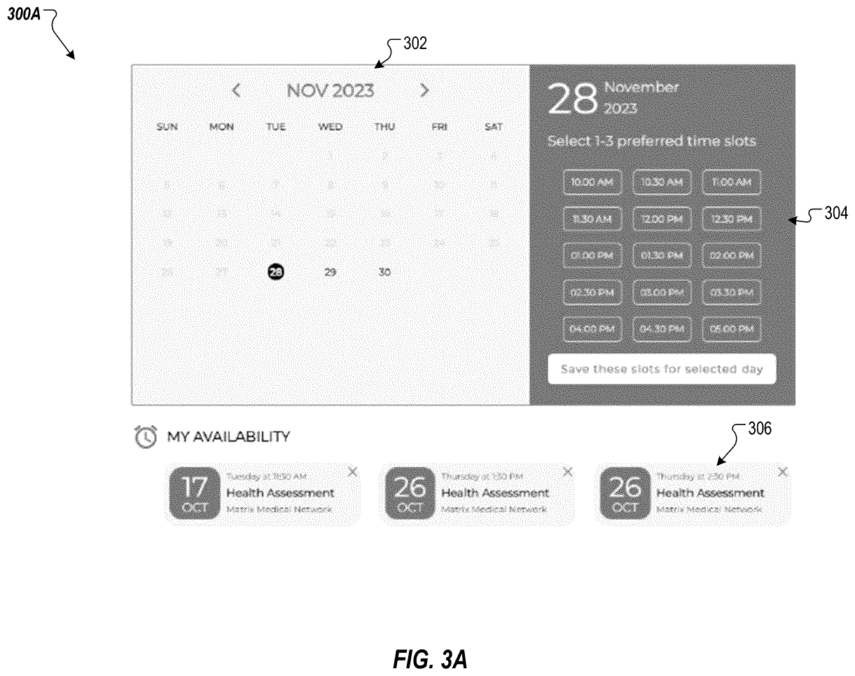

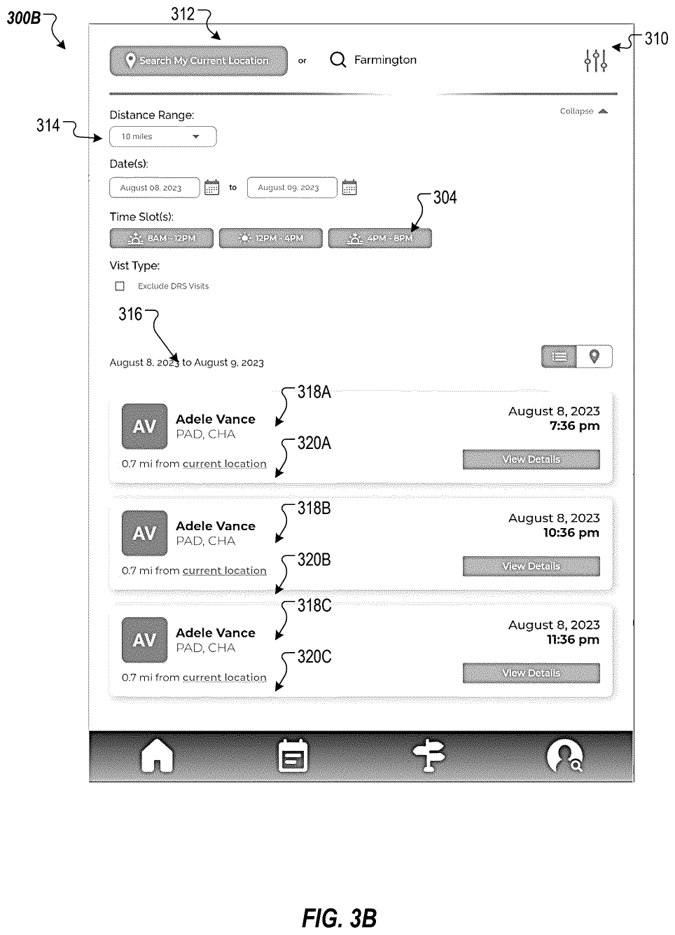

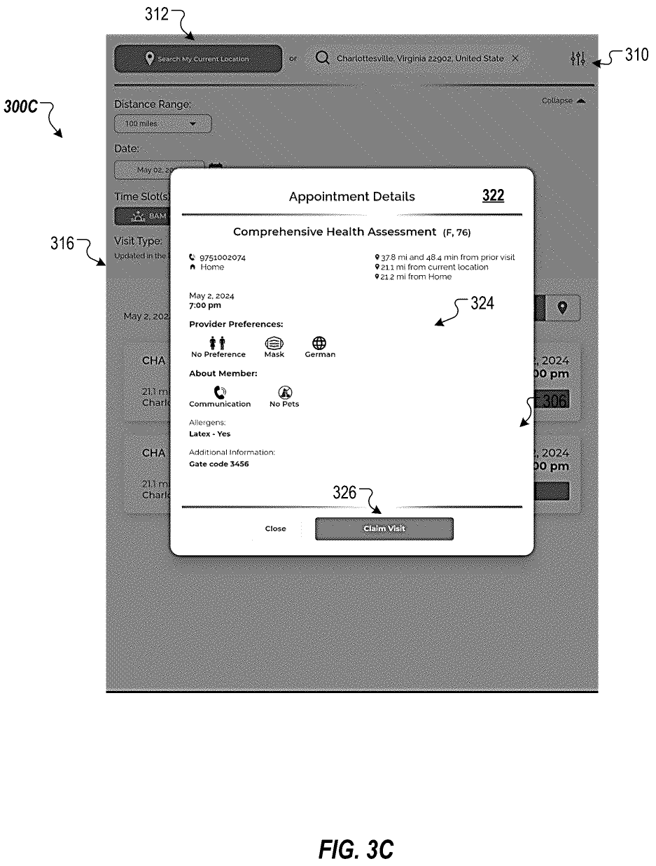

FIG. 3A depicts an example of a display panel 300A for data item selection, in accordance with implementations of the present disclosure. The display panel 300A can be generated by a scheduling system (e.g., the scheduling system 112 described with reference to FIGS. 1A and 1B) and can be displayed by a user device (e.g., the user device 104A described with reference to FIGS. 1A and 1B) of a user identified as a potential service provider. The display panel 300A can include a display of a calendar portion 302 (e.g., a month or a week) and a list of potential time slots 304 that can be selected by the user per day or within a time range (including a starting day and an end day). A portion 306 of the display panel 300A can include a list of previously selected availability for providing services.

FIG. 3B depicts an example display panel 300B of action selection that can be executed in accordance with implementations of the present disclosure. The display panel 300B can be generated by a scheduling system (e.g., the scheduling system 112 described with reference to FIGS. 1A and 1B) and can be displayed by a user device (e.g., the user device 104A described with reference to FIGS. 1A and 1B) of a user identified as a potential service provider. The display panel 300B can be displayed by the user device (e.g., the user device 104A described with reference to FIGS. 1A and 1B) to enable the user to generate a user input indicating a selection of an event. The display panel 300B can include a display of a geographical filter portion 310 that is applicable to selected time slots 304 within particular time intervals (e.g., day range). The geographical filter portion 310 can include a selection of a starting point 312 and a distance range 314 (e.g., defined as a transportation or driving duration). The display panel 300B can include a list 316 of selected users 318A, 318B, 318C requesting services, the users being within a geographical boundary at a respective distance 320A, 320B, 320C from the location 312 of the user providing services.

FIG. 3C depicts an example display panel 300C of a selectable action that can be displayed in accordance with implementations of the present disclosure. The display panel 300C can be generated by a scheduling system (e.g., the scheduling system 112 described with reference to FIGS. 1A and 1B) and can be displayed by a user device (e.g., the user device 104A described with reference to FIGS. 1A and 1B) of a user identified as a potential service provider. The display panel 300C can include a pop-up window 322 that overlaps a display of a geographical filter portion 310 and the list 316 of selected users. The pop-up window 322 can include information 324 defining details of requested service and a selection option 326 to enter a user input indicating an acceptance (e.g., claim) of the displayed requested service.

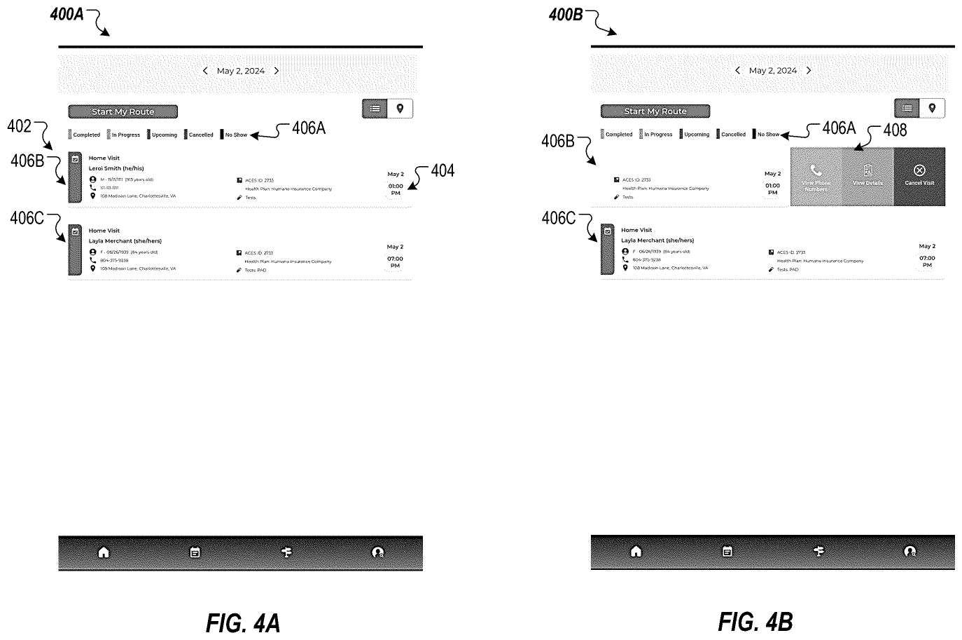

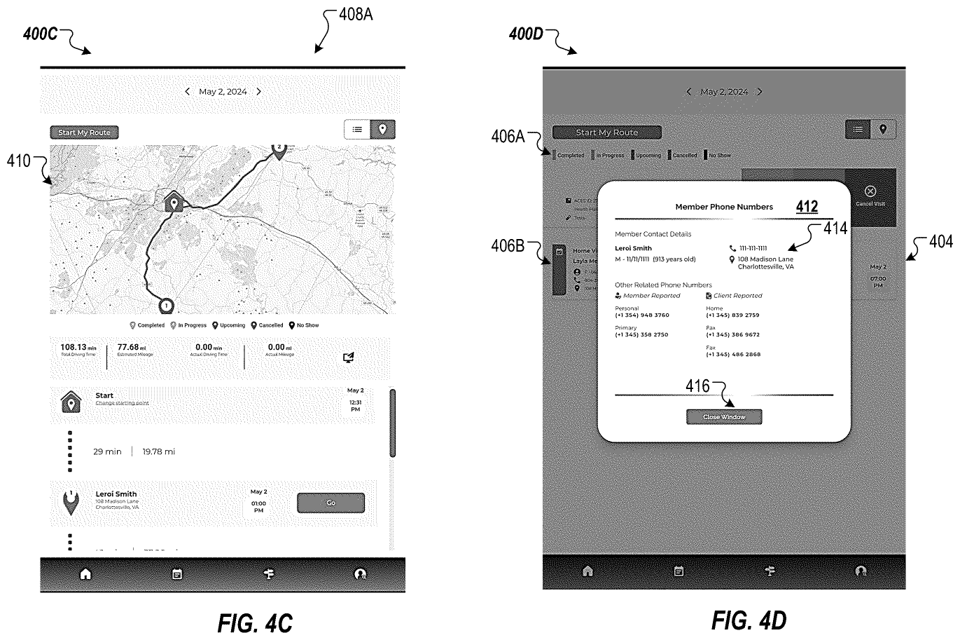

FIG. 4A depicts an example display panel 400A of action sequences that can be executed in accordance with implementations of the present disclosure. The display panel 400A can be generated by a scheduling system (e.g., the scheduling system 112 described with reference to FIGS. 1A and 1B) in response to receiving, from a user device, an inquiry to render the user interface with the first visual representations of upcoming events. The display panel 400A can be displayed by the user device (e.g., the user device 104A described with reference to FIGS. 1A and 1B) to enable the user to select from a list of upcoming visits 402, an upcoming event for respective route display. The display panel 400A can include an example of an interactive interface for selection of actions based on a time 404 of the scheduled service. The display panel 400A can be updated in real time to include real time indicators 406A, 406B, 406C of service statuses.

FIG. 4B depicts an example display panel 400B of route planning for resource allocation actions that can be executed in accordance with implementations of the present disclosure. The display panel 400B can be generated by a scheduling system (e.g., the scheduling system 112 described with reference to FIGS. 1A and 1B) in response to a selection of a scheduled service, as shown in FIG. 4A. The display panel 400B can be displayed by a user device (e.g., the user device 104A described with reference to FIGS. 1A and 1B) to provide access to a selection of actions 408 associated with a selected scheduled service. The actions 408 can include initiation of communication between user devices, retrieval of scheduled service data and scheduled service cancellation.

FIG. 4C depicts an example display panel of route mapping for resource allocation actions that can be executed in accordance with implementations of the present disclosure. The display panel 400B can be generated by a scheduling system (e.g., the scheduling system 112 described with reference to FIGS. 1A and 1B) in response to a selection of a scheduled service, as shown in FIG. 4A or FIG. 4B. The display panel 400C can be displayed by a user device (e.g., the user device 104A described with reference to FIGS. 1A and 1B). The display panel 400C can include a map 410 to assist the user providing services to travel to a selected user. The display panel 400C can be updated in real time to display action data, related to an ongoing action selected from the sequence of actions.

FIG. 4D depicts an example display panel 400D of communication selection during route planning for resource allocation actions that can be executed in accordance with implementations of the present disclosure. The display panel 400D can be generated by a scheduling system (e.g., the scheduling system 112 described with reference to FIGS. 1A and 1B) and can be displayed by a user device (e.g., the user device 104A described with reference to FIGS. 1A and 1B) of a user identified as a potential service provider. The display panel 400D can include a pop-up window 412 that overlaps a display of the time 404 of the scheduled service and of the real time indicators 406A, 406B, 406C of service statuses. The pop-up window 412 can include information 414 defining details of the scheduled service and a selection option 416 to enter a user input to close the pop-up window 412.

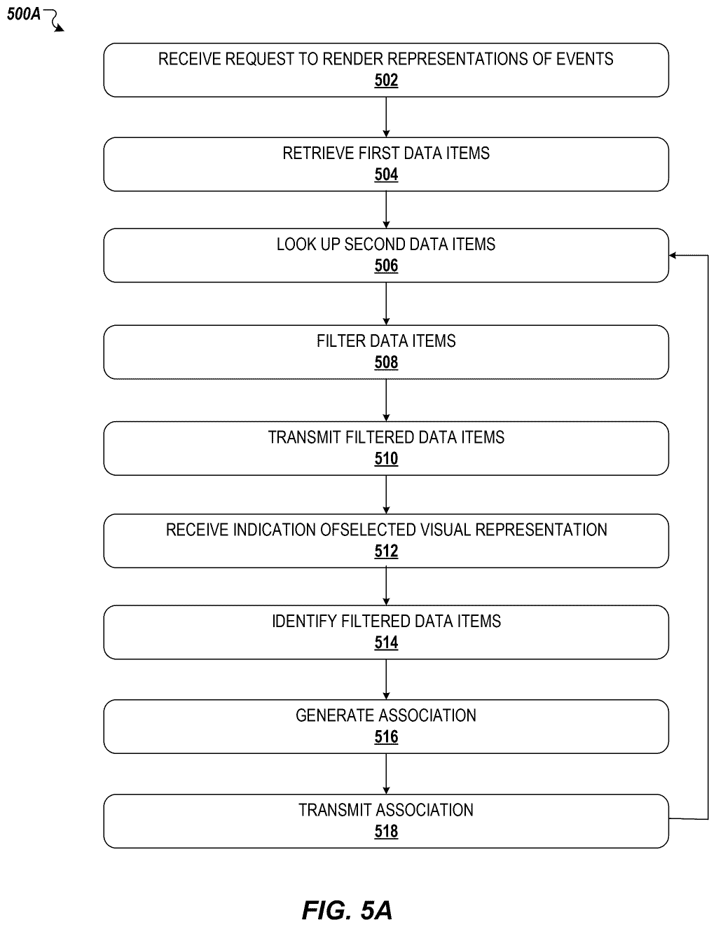

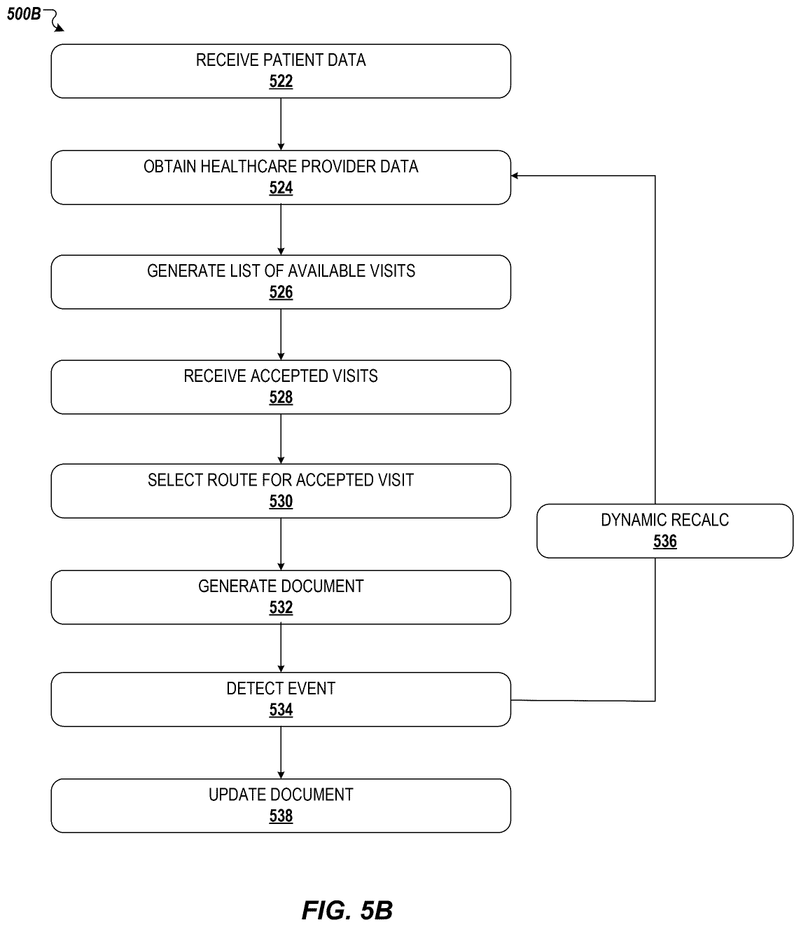

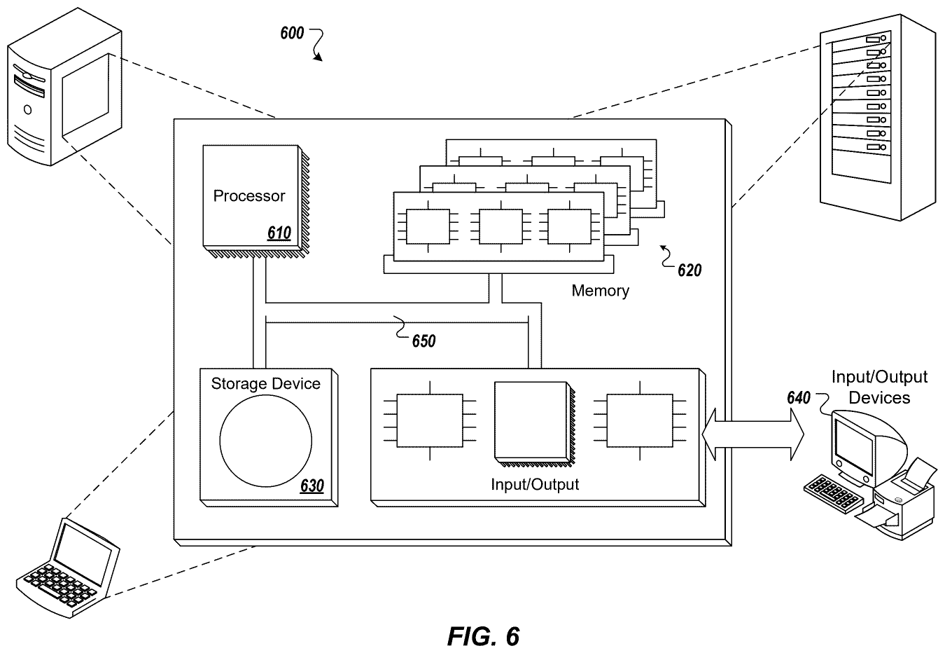

FIG. 5A depicts an example remediation process 500A that can be executed in accordance with implementations of the present disclosure. In some examples, the example process 500A is provided using one or more computer-executable programs executed by one or more computing devices, such as a scheduling system 112 of the private cloud system 102 described with reference to FIGS. 1A and 1B or an example computing system 600 described with reference to FIG. 6.

At 502, a request to render representations of events on a graphical user interface of a user device (e.g., user device 104A described with reference to FIGS. 1A and 1B) is received. The graphical user interface is generated by a user interface module. The graphical user interface renders first visual representations of requested medical visits and second visual representations of real time updates for accepted visits. The request specifies an identifier of a first user associated with the user device. The request can include a geographic location of the user device that can be automatically detected by a sensor included in the user device. The first user associated with the user device can be a service provider, such as a healthcare practitioner, a therapist, a beautician, or any other type of service provider associated with a service providing network.

At 504, first data items are retrieved from a database (e.g., 108A described with reference to FIGS. 1A and 1B), by one or more processors (e.g., a processor of the user device or a processor of a server system, as described with reference to FIGS. 1A and 1B). The first data items can specify one or more qualifications of the first user to perform services (e.g., a particular type of service types).

At 506, second data items are identified using a look up action executed, by the one or more processors. The second data items include information related to one or more second users requesting services. The second data items include information specifying one or more attributes of the second users. The second data items can be labeled with an indicator representing a geographic location of requested services. The second users can be service recipients, such as patients, clients, customers, or any other type of service recipients associated with a service providing network.

At 508, data items are filtered, by the one or more processors, by matching the attributes of the second users with the qualifications of the first user. The filtering includes identification the geographic location in the request within a radius around the geographic location of requested services.

At 510, filtered data items can be encrypted for transmission and transmitted for display to the graphical user interface of the user device. The user interface module is configured to generate, based on the filtered data items, the requested user interface with first visual representations of requested medical visits in the requested geographic location. Each first visual representation is selectable to indicate selection of a requested medical visit represented by the first visual representation.

At 512, an indication of selected visual representation of a data item is received, by the one or more processors. The indication specifies the identifier of the first user of the user device.

At 514, filtered data items are identified, by the one or more processors.

At 516, an association is generated, by the one or more processors, between the one or more first data items associated with the identifier of the first user associated with the user device and the identified data item. The association specifies that a requested service represented by the identified data item is an accepted visit of the first user represented by the identifier associated with the one or more first data items.

At 518, the association is transmitted, by the one or more processors to a database for storage. The stored association is used to traverse the data items stored in the memory to identify the association between the one or more first data items and the identified data item. The user interface, rendered on the user device, is updated with a second visual representation representing the accepted visit, based on the identified association. The second visual representation is rendered in real-time relative to the indication of the selection of the first visual representation.

FIG. 5B depicts an example remediation process 500B that can be executed in accordance with implementations of the present disclosure. In some examples, the example process 500B is provided using one or more computer-executable programs executed by one or more computing devices, such as a scheduling system 112 of the private cloud system 102 described with reference to FIGS. 1A and 1B or an example computing system 600 described with reference to FIG. 6.

At 522, patient data is received, by one or more processors from a database. The patient data can include patient identifiers, existing conditions (known allergies, previous diagnosis, ongoing medication), medical history, requested (e.g., scheduled) tests, symptoms, reasons for visit request, and other data that can affect a visit selection.

At 524, healthcare provider data is obtained, by the one or more processors from a database. The healthcare provider data can include one or more healthcare provider locations, healthcare provider identifiers, qualifications, experience, available diagnosing tools and equipment. The healthcare provider data can include time constraints and geographic restrictions of the health care provider.

At 526, a list of visits available for the healthcare provider is generated, by the one or more processors. The list of visits available for the healthcare provider is generated by filtering from the visits requested by the patients, a subset of visits with matching criteria that are within a geographic radius around the healthcare provider locations. The matching criteria can include matching of patient data (e.g., patient conditions and requested tests) with the healthcare provider data (e.g., qualifications, experience, available diagnosing tools and equipment) to facilitate accuracy of provided service and completeness of medical visit.

At 528, a user input including one or more accepted visits are received, by the one or more processors. The user input can include identifiers of the accepted visits, with respective locations dates and times. The user input can be entered through an interaction with the display panel, as described with reference to FIGS. 3A-3C and 4A-4D.

At 530, a route for an accepted visit is generated and selected, by the one or more processors. The route can be automatically generated to assist a transportation of the healthcare provider to the accepted visit. In some implementations, route conditions are continuously monitored by a vehicle connected to the user device that transport the health care provider to allocation of the accepted visit. The route can be updated based on route conditions. The update of the route can be compared to healthcare provider data defining time and geographic restrictions of the health care provider.

At 532, a document corresponding to the accepted visit is generated, by the one or more processors. The document can include a summary of patient data relevant to the accepted visit, identifiers of a status of accepted visits, and newly added information indicative of an outcome of the accepted visit, as described with reference to FIGS. 3A-3C and 4A-4D.

At 534, an impeding event affecting the accepted event (e.g., visit) is detected, by the one or more processors. The impeding event detection can include processing of sensor data, detected by a sensor of a vehicle used for transportation of the healthcare provider to the accepted visit. The event detection can include user input describing the event. The user input can be transmitted by a user device of the patient or the healthcare provider. The event can include one or more conditions (e.g., occurrence of one or more factors, such as unusual traffic, accidents, emergencies, etc.) preventing the successful completion of the medical visit.

At 536, a dynamic recalculation of data related to accepted visits is performed, by the one or more processors. The dynamic recalculation can include application of a remediation rule. The remediation rule can define generation of remediation plans including a replacement remediation action defining placeholder parameters and conditions for replacing conditions preventing the successful completion of the medical visit with substitute conditions to remedy the event. The remediation rule definitions include an action name, an action description, and a script that defines the replacement action. A single remediation action can be included in each rule. The remediation rule definitions include rerouting the healthcare provider along a different route or replacing the accepted visit with another visit to remedy the detected event. The remediation rule definitions can be mapped to the data tables using a mapping that can be stored in a database. The mapping defines associations between event types, the remediation rule definitions, and healthcare provider restrictions.

The remediation plans can be selected using a prediction model. In some example implementations, the machine learning model can be subjected to supervised pre-training, for example, to perform a selection of remediation plans for interrelated data tables. The machine learning model can be fine-tuned to optimize the sequence of service (and resource) allocation actions within the remediation plans to minimize a risk of visit failure (e.g., visit cancellation), by using minimal of processing resources, and by avoiding a breach of healthcare provider restrictions. The fine-tuning of the first machine learning model can include adjusting weights applied to service allocation actions relative to event types. For example, the weights applied by the machine learning model can be adjusted through backpropagation of the error (or another optimization technique) present in the data tables. As noted, the weights applied by the machine learning model can be adjusted during a temporary fine-tuning, the weights applied by the machine learning model can remain static to prevent drift and unstable behavior (e.g., loss oscillations and/or the like) after the error of the prediction drops below a particular threshold. The verification of the error can be performed according to a set frequency, and the readjustment of the weights can be reimplemented at any time the error level exceeds the acceptable error level. The optimization provided by the predictive models allows private cloud deployments to keep resource capacity at a minimum to save costs. The remediation plans include a remediation workflow listing a sequence of applicable resource allocation actions mapped to the one or more geographic regions with overlapping sections. The resource allocation actions can include any type of service providing actions that can be performed by qualified personnel, such as medical visits. The remediation plan can be derived from one or more remediation rule definitions to correct the erroneous entries in each of the one or more data tables. The remediation plans can include generating a recommendation comprising a permutation of a traversal of the second data items; and generating a notification for displaying the recommendation. In some implementations, a remediation plan includes generating, by the one or more processors, a signal to redirect the health care provider to a selected patient. The signal can trigger a display of an updated map (e.g., map 410, as described with reference to FIG. 4C) and/or an automatic redirection of a vehicle (e.g., an autonomous vehicle 130, as described with reference to FIG. 1B) of the health care provider to a selected patient.

At 538, the document corresponding to the accepted visit is updated, by the one or more processors. The document corresponding to the accepted visit is updated to include a summary of actions (including remediation actions) executed in association with the accepted visit.

The example processes 500A and 500B advantageously enable optimized execution of service selection and assistance in completing services, including remediation of impeding events affecting accepted events. The example processes 500A and 500B provide automated mechanisms to identify remediation plans optimized for correction of events. Without an automated mechanism to identify remediation plans, the task can be very cumbersome and time-consuming due to the interrelation between multiple service requests, service allocation, and service provider restrictions. Identification and correction of actions in view of detected impeding events, is essential to enable successful completion of accepted events. The execution of remediation across overlapping geographic regions complying with requirements can be improved by automatically executing validation operations.

Referring now to FIG. 6, a schematic diagram of an example computing system 600 is provided. The system 600 can be used for the operations described in association with the implementations described herein. For example, the system 600 can be included in any or all of the server components discussed herein, such as the components of the example system 100A, 100B described with reference to FIGS. 1A and 1B. The system 600 includes a processor 610, a memory 620, a storage device 630, and an input/output device 640. The components 610, 620, 630, 640 are interconnected using a system bus 650. The processor 610 is capable of processing instructions for execution of processes (e.g., example process 300 described with reference to FIG. 3) within the system 600. In some implementations, the processor 610 is a single-threaded processor. In some implementations, the processor 610 is a multi-threaded processor. The processor 610 is capable of processing instructions stored in the memory 620 or on the storage device 630 to display graphical information for a user interface on the input/output device 640.

The memory 620 stores information within the system 600. In some implementations, the memory 620 is a computer-readable medium. In some implementations, the memory 620 is a volatile memory unit. In some implementations, the memory 620 is a non-volatile memory unit. The storage device 630 is capable of providing mass storage for the system 600. In some implementations, the storage device 630 is a computer-readable medium. In some implementations, the storage device 630 can be a floppy disk device, a hard disk device, an optical disk device, or a tape device. The input/output device 640 provides input/output operations for the system 600. In some implementations, the input/output device 640 includes a keyboard and/or pointing device. In some implementations, the input/output device 640 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier (e.g., in a machine-readable storage device, for execution by a programmable processor), and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a particular activity or bring about a particular result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random-access memory or both. Elements of a computer can include a processor for executing instructions and one or more memories for storing instructions and data. Generally, a computer can also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, for example, a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other implementations are within the scope of the following claims.

A number of implementations of the present disclosure have been described. Nevertheless, it will be understood that various modifications can be made without departing from the spirit and scope of the present disclosure. Accordingly, other implementations are within the scope of the following claims.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application.

Example 1. A system for communicating with a user device and causing rendering of one or more user interfaces on the user device, the system comprising: a memory storing first data items and second data items; a user interface module that generates a user interface that renders first visual representations of upcoming events and second visual representations of real time updates for accepted upcoming events; and a processor in communication with the memory configured to perform operations comprising: receiving, from a user device, an inquiry to render the user interface with the first visual representations of upcoming events, the inquiry specifying an identifier of a first user associated with the user device and the inquiry specifying a location of the user device; retrieving, from the memory, one or more first data items associated with the identifier of the first user associated with the user device transmitting the inquiry, with the one or more first data items specifying one or more qualifications of the first user; looking up, in the memory, the second data items, each of the second data items being labeled with an indicator representing a location, the second data items representing upcoming events of second users, and each of the second data items specifying one or more attributes of a respective second user; filtering the second data items to identify filtered data items by matching the one or more attributes with the one or more qualifications relative to the location; transmitting, to the user interface module, the filtered data items, to generate the user interface comprising first visual representations of upcoming events within the location; receiving, through the user interface from the user device, an indication of a selected visual representation from the first visual representations, the indication specifying the identifier of the first user of the user device; identifying a portion of the filtered data items represented by the selected visual representation; generating, in memory, an association between the portion of the filtered data and the identifier of the first user, the association specifying that an accepted event of the first user represented by the identifier associated with the one or more first data items; and transmitting, to the user interface module, the association specifying the accepted event, to traverse the second data items stored in the memory to automatically update the user interface rendered on the user device with a second visual representation representing the accepted event, the second visual representation being rendered in real-time relative to the indication of the selected visual representation.

Example 2. The system of the preceding example, wherein the one or more first data items comprise medical qualifications and availability calendars relative to the location.

Example 3. The system of any of the preceding examples, wherein the one or more second data items comprise medical records and appointment zone data.

Example 4. The system of any of the preceding examples, wherein matching the one or more attributes with the one or more qualifications relative to the location comprises mapping the medical records and the medical qualifications relative to zone mapping based on the location.

Example 5. The system of any of the preceding examples, wherein the mapping defines associations between the appointment zone data and the location that are compatible with the availability calendars.

Example 6. The system of any of the preceding examples, wherein the mapping comprises executing a geo-dialing algorithm determining distances and estimated travel times between the appointment zone data and the location.

Example 7. The system of any of the preceding examples, wherein the respective available upcoming events are ranked based on a timeline.

Example 8. The system of any of the preceding examples, wherein the operations further comprise: receiving event data of an impeding event affecting the accepted event; and updating the user interface to display corrected entries indicative of the impeding event.

Example 9. The system of any of the preceding examples, wherein the impeding event affecting the accepted event comprises a traffic event associated with a predicted failure to complete the accepted event by the first user.

Example 10. The system of any of the preceding examples, wherein the operations further comprise: identifying a remedy to compensate for the impeding event; generating a recommendation comprising a permutation of a traversal of the second data items; and generating a notification for displaying the recommendation.

Example 11. The system of any of the preceding examples, wherein the filtered data items are encrypted for transmission.

Example 12. A computer-implemented method comprising: receiving, from a user device, an inquiry to render a user interface with the first visual representations of upcoming events, the inquiry specifying an identifier of a first user associated with the user device and the inquiry specifying a location of the user device; retrieving, from a memory, one or more first data items associated with the identifier of the first user associated with the user device transmitting the inquiry, with the one or more first data items specifying one or more qualifications of the first user; looking-up, in the memory, the second data items, each of the second data items being labeled with an indicator representing a location, the second data items representing upcoming events of second users, and each of the second data items specifying one or more attributes of a respective second user; filtering the second data items to identify filtered data items by matching the one or more attributes with the one or more qualifications relative to the location; transmitting, to the user interface module, the filtered data items, to generate the user interface comprising first visual representations of upcoming events within the location; receiving, through the user interface from the user device, an indication of a selected visual representation from the first visual representations, the indication specifying the identifier of the first user of the user device; identifying a portion of the filtered data items represented by the selected visual representation; generating, in memory, an association between the portion of the filtered data and the identifier of the first user, the association specifying that an accepted event of the first user represented by the identifier associated with the one or more first data items; and transmitting, to the user interface module, the association specifying the accepted event, to traverse the second data items stored in the memory to automatically update the user interface rendered on the user device with a second visual representation representing the accepted event, the second visual representation being rendered in real-time relative to the indication of the selected visual representation.

Example 13. The computer-implemented method of the preceding example, wherein the one or more first data items comprise medical qualifications and availability calendars relative to the location.

Example 14. The computer-implemented method of any of the preceding examples, wherein the one or more second data items comprise medical records and appointment zone data.

Example 15. The computer-implemented method of any of the preceding examples, wherein matching the one or more attributes with the one or more qualifications relative to the location comprises mapping the medical records and the medical qualifications relative to zone mapping based on the location.

Example 16. The computer-implemented method of any of the preceding examples, wherein the mapping defines associations between the appointment zone data and the location that are compatible with the availability calendars, wherein the mapping comprises executing a geo-dialing algorithm determining distances and estimated travel times between the appointment zone data and the location.

Example 17. The computer-implemented method of any of the preceding examples, wherein the respective available upcoming events are ranked based on a timeline.

Example 18. The computer-implemented method of any of the preceding examples, further comprising: receiving event data of an impeding event affecting the accepted event; updating the user interface to display corrected entries indicative of the impeding event, wherein the impeding event affecting the accepted event comprises a traffic event associated with a predicted failure to complete the accepted event by the first user; identifying a remedy to compensate for the impeding event; generating a recommendation comprising a permutation of a traversal of the second data items; and generating a notification for displaying the recommendation.

Example 19. The computer-implemented method of any of the preceding examples, wherein the filtered data items are encrypted for transmission.

Example 20. A non-transitory computer-readable media encoded with a computer program, the computer program comprising instructions that when executed by one or more computers cause the one or more computers to perform operations comprising: receiving, from a user device, an inquiry to render a user interface with the first visual representations of upcoming events, the inquiry specifying an identifier of a first user associated with the user device and the inquiry specifying a location of the user device; retrieving, from a memory, one or more first data items associated with the identifier of the first user associated with the user device transmitting the inquiry, with the one or more first data items specifying one or more qualifications of the first user; looking-up, in the memory, the second data items, each of the second data items being labeled with an indicator representing a location, the second data items representing upcoming events of second users, and each of the second data items specifying one or more attributes of a respective second user; filtering the second data items to identify filtered data items by matching the one or more attributes with the one or more qualifications relative to the location; transmitting, to the user interface module, the filtered data items, to generate the user interface comprising first visual representations of upcoming events within the location; receiving, through the user interface from the user device, an indication of a selected visual representation from the first visual representations, the indication specifying the identifier of the first user of the user device; identifying a portion of the filtered data items represented by the selected visual representation; generating, in memory, an association between the portion of the filtered data and the identifier of the first user, the association specifying that an accepted event of the first user represented by the identifier associated with the one or more first data items; and transmitting, to the user interface module, the association specifying the accepted event, to traverse the second data items stored in the memory to automatically update the user interface rendered on the user device with a second visual representation representing the accepted event, the second visual representation being rendered in real-time relative to the indication of the selected visual representation.

What is claimed is:

1. A system for communicating with a user device and causing rendering of one or more user interfaces on the user device, the system comprising:
a memory storing first data items and second data items;
a user interface module that generates a user interface that renders first visual representations of upcoming events and second visual representations of real time updates for accepted upcoming events; and
a processor in communication with the memory configured to perform operations comprising:
receiving, from a user device, an inquiry to render the user interface with the first visual representations of upcoming events, the inquiry specifying an identifier of a first user associated with the user device and the inquiry specifying a location of the user device;
retrieving, from the memory, one or more first data items associated with the identifier of the first user associated with the user device transmitting the inquiry, with the one or more first data items specifying one or more qualifications of the first user;
looking up, in the memory, the second data items, each of the second data items being labeled with an indicator representing a location, the second data items representing upcoming events of second users, and each of the second data items specifying one or more attributes of a respective second user;
filtering the second data items to identify filtered data items by matching the one or more attributes with the one or more qualifications relative to the location;
transmitting, to the user interface module, the filtered data items, to generate the user interface comprising first visual representations of upcoming events within the location;
receiving, through the user interface from the user device, an indication of a selected visual representation from the first visual representations, the indication specifying the identifier of the first user of the user device;
identifying a portion of the filtered data items represented by the selected visual representation;
generating, in memory, an association between the portion of the filtered data and the identifier of the first user, the association specifying that an accepted event of the first user represented by the identifier associated with the one or more first data items; and
transmitting, to the user interface module, the association specifying the accepted event, to traverse the second data items stored in the memory to automatically update the user interface rendered on the user device with a second visual representation representing the accepted event, the second visual representation being rendered in real-time relative to the indication of the selected visual representation.

2. The system of claim 1, wherein the one or more first data items comprise medical qualifications and availability calendars relative to the location.

3. The system of claim 2, wherein the one or more second data items comprise medical records and appointment zone data.

4. The system of claim 3, wherein matching the one or more attributes with the one or more qualifications relative to the location comprises mapping the medical records and the medical qualifications relative to zone mapping based on the location.

5. The system of claim 4, wherein the mapping defines associations between the appointment zone data and the location that are compatible with the availability calendars.

6. The system of claim 5, wherein the mapping comprises executing a geo-dialing algorithm determining distances and estimated travel times between the appointment zone data and the location.

7. The system of claim 1, wherein the respective available upcoming events are ranked based on a timeline.

8. The system of claim 1, wherein the operations further comprise:
receiving event data of an impeding event affecting the accepted event; and
updating the user interface to display corrected entries indicative of the impeding event.

9. The system of claim 8, wherein the impeding event affecting the accepted event comprises a traffic event associated with a predicted failure to complete the accepted event by the first user.

10. The system of claim 9, wherein the operations further comprise:
identifying a remedy to compensate for the impeding event;
generating a recommendation comprising a permutation of a traversal of the second data items; and
generating a notification for displaying the recommendation.

11. The system of claim 1, wherein the filtered data items are encrypted for transmission.

12. A computer-implemented method comprising:
receiving, from a user device, an inquiry to render a user interface with the first visual representations of upcoming events, the inquiry specifying an identifier of a first user associated with the user device and the inquiry specifying a location of the user device;
retrieving, from a memory, one or more first data items associated with the identifier of the first user associated with the user device transmitting the inquiry, with the one or more first data items specifying one or more qualifications of the first user;
looking-up, in the memory, the second data items, each of the second data items being labeled with an indicator representing a location, the second data items representing upcoming events of second users, and each of the second data items specifying one or more attributes of a respective second user;
filtering the second data items to identify filtered data items by matching the one or more attributes with the one or more qualifications relative to the location;
transmitting, to the user interface module, the filtered data items, to generate the user interface comprising first visual representations of upcoming events within the location;
receiving, through the user interface from the user device, an indication of a selected visual representation from the first visual representations, the indication specifying the identifier of the first user of the user device;
identifying a portion of the filtered data items represented by the selected visual representation;
generating, in memory, an association between the portion of the filtered data and the identifier of the first user, the association specifying that an accepted event of the first user represented by the identifier associated with the one or more first data items; and
transmitting, to the user interface module, the association specifying the accepted event, to traverse the second data items stored in the memory to automatically update the user interface rendered on the user device with a second visual representation representing the accepted event, the second visual representation being rendered in real-time relative to the indication of the selected visual representation.

13. The computer-implemented method of claim 12, wherein the one or more first data items comprise medical qualifications and availability calendars relative to the location.

14. The computer-implemented method of claim 13, wherein the one or more second data items comprise medical records and appointment zone data.

15. The computer-implemented method of claim 14, wherein matching the one or more attributes with the one or more qualifications relative to the location comprises mapping the medical records and the medical qualifications relative to zone mapping based on the location.

16. The computer-implemented method of claim 15, wherein the mapping defines associations between the appointment zone data and the location that are compatible with the availability calendars, wherein the mapping comprises executing a geo-dialing algorithm determining distances and estimated travel times between the appointment zone data and the location.

17. The computer-implemented method of claim 12, wherein the respective available upcoming events are ranked based on a timeline.

18. The computer-implemented method of claim 12, further comprising:
receiving event data of an impeding event affecting the accepted event;
updating the user interface to display corrected entries indicative of the impeding event, wherein the impeding event affecting the accepted event comprises a traffic event associated with a predicted failure to complete the accepted event by the first user;
identifying a remedy to compensate for the impeding event;
generating a recommendation comprising a permutation of a traversal of the second data items; and
generating a notification for displaying the recommendation.

19. The computer-implemented method of claim 12, wherein the filtered data items are encrypted for transmission.

20. A non-transitory computer-readable media encoded with a computer program, the computer program comprising instructions that when executed by one or more computers cause the one or more computers to perform operations comprising:
receiving, from a user device, an inquiry to render a user interface with the first visual representations of upcoming events, the inquiry specifying an identifier of a first user associated with the user device and the inquiry specifying a location of the user device;
retrieving, from a memory, one or more first data items associated with the identifier of the first user associated with the user device transmitting the inquiry, with the one or more first data items specifying one or more qualifications of the first user;
looking-up, in the memory, the second data items, each of the second data items being labeled with an indicator representing a location, the second data items representing upcoming events of second users, and each of the second data items specifying one or more attributes of a respective second user;
filtering the second data items to identify filtered data items by matching the one or more attributes with the one or more qualifications relative to the location;
transmitting, to the user interface module, the filtered data items, to generate the user interface comprising first visual representations of upcoming events within the location;
receiving, through the user interface from the user device, an indication of a selected visual representation from the first visual representations, the indication specifying the identifier of the first user of the user device;
identifying a portion of the filtered data items represented by the selected visual representation;
generating, in memory, an association between the portion of the filtered data and the identifier of the first user, the association specifying that an accepted event of the first user represented by the identifier associated with the one or more first data items; and
transmitting, to the user interface module, the association specifying the accepted event, to traverse the second data items stored in the memory to automatically update the user interface rendered on the user device with a second visual representation representing the accepted event, the second visual representation being rendered in real-time relative to the indication of the selected visual representation.
